(19) United States
(12) Patent Application Publication
(10) Pub. No.: US 2026/0157753 A1
(43) Pub. Date: Jun. 11, 2026

(54) STAPLES, INSERTION DEVICES, AND ASSOCIATED METHODS

(71) Applicant: NOVASTEP, Rennes (FR)

(72) Inventors: Axel CREMER, Fahrenkrug (DE); Torben BRÖHAN, Rottweil (DE); Henrik NÜSSE, Kronshagen (DE); Thomas CHEVREL, Rennes (FR)

(73) Assignee: NOVASTEP, Rennes (FR)

(21) Appl. No.: 18/707,211

(22) PCT Filed: Aug. 2, 2023
(86) PCT No.: PCT/EP2023/071397

§ 371 Date: May 3, 2024

Related U.S. Application Data
(60) Provisional application No. 63/394,839, filed on Aug. 3, 2022.

(51) Int. Cl. A61B 17/064; A61B 17/10

(52) U.S. Cl. CPC A61B 17/0642; A61B 17/10

(57) ABSTRACT

Staples, insertion devices, and associated methods are described. The invention relates to a bone staple tool (3) comprising: a main body (4) having a main body distal end; an intermediate body (5) having an intermediate body proximal end (52) and an intermediate body distal end (54) at least partially disposed within the main body at the intermediate body proximal end, the intermediate body (5) extending from the main body having a slit (5024); and a gripping member (6) that extends from the intermediate body distal end (54) having a first member (62) and a second member (64) defining a channel (66) therebetween.

This application claims the benefit of and priority to U.S. Provisional Application, entitled “Staples, Insertion Devices, and Associated Methods”, filed on Aug. 3, 2022 and having application Ser. No. 63/394,839, the entirety of said application being incorporated herein by reference.

### FIELD

The disclosure relates to the field of medical devices and medical instruments. More particularly, the disclosure relates to compression staples useful in the repair of fractured bones and inserter instruments for implanting compression staples in bones.

### BACKGROUND

Intramedullary Fracture fixation with compression staple is common for reconstruction of the forefoot, midfoot and rearfoot. Traditionally staples are delivered separately to their instruments and are loaded on the instruments during the reconstruction surgical procedure. More recently, staples have also been supplied preloaded on the inserter instrument, making the staples ready for implantation directly at the time of need. Preloaded staples typically have a support member that extends under the bridge of the staple to achieve parallel legs of the staple during implantation. While this structural arrangement enables the preloading of the staple in the instrument, it has the drawback of eliminating the ability of the staple to be inserted directly flush with the bone and necessitates another step during the surgery to achieve the desired flush final positioning. Furthermore, the inserter must be separated from the staple by a twist or push to the side of the staple, which requires significant space in the often limited surgical field. The maneuver is awkward for the surgeon, and can require placing force on adjacent tissues to achieve full disengagement of the instrument and the staple.

A need remains, therefore, for improved surgical staples, including orthopedic compression staples, insertion devices for implanting staples, and associated methods, such as methods of implanting staples, including compression staples.

### SUMMARY

Various example surgical staples, including orthopedic compression staples, insertion devices, and associated methods, including methods of implanting staples, are described.

Additional understanding of the inventive staples, insertion devices, and associated methods can be obtained by reviewing the detailed description of selected examples, below, with reference to the appended drawings.

A compression staple and staple inserter are provided for corrections of bone deformities and treatments of foot joint stiffness. The disclosure is particularly useful for, but not limited to, treatments of the forefoot (such as Akin osteotomy, Bunion, MTP arthrodesis etc.), midfoot (such as Navicular cuneiform arthrodesis, Lisfranc arthrodesis leg, Lapidus Procedure etc.) and rearfoot (such as Talo-Navicular arthrodesis, Calcaneo-cuboid arthrodesis etc.).

In accordance with the present invention, a bone staple tool comprises a main body having a main body distal end; an intermediate body having an intermediate body proximal end and an intermediate body distal end at least partially disposed within the main body at the intermediate body proximal end, the intermediate body extending from the main body having a slit that extends from the main body distal end to the intermediate body distal end; and a gripping member that extends from the intermediate body distal end having a first member and a second member defining a channel therebetween.

The bone staple tool wherein a width of the main body is larger than a width of the intermediate body.

The bone staple tool wherein the channel is substantially centered at the slit.

The bone staple tool wherein the first member and the second member of the gripping member have protrusions configured to retain a staple within the channel.

The bone staple tool wherein the gripping member is configured to receive a staple no further than a staple bridge top portion.

The bone staple tool further comprising a mechanism for retracting the protrusions.

The bone staple tool wherein the main body has a grip.

The bone staple tool wherein a width of the gripping member is larger than a width of the intermediate body and the main body.

The bone staple tool wherein the channel has a channel width that is smaller than a head of a staple.

The bone staple tool wherein the intermediate body is a metal.

The bone staple tool wherein the main body is a polymer.

The bone staple tool wherein the gripping member is a metal.

The bone staple tool wherein the gripping member has a gripping member distal end and a gripping member proximal end, and the gripping member proximal end has a width more narrow than the gripping member distal end.

The bone staple tool wherein the intermediate body and the gripping member are made of the same material.

The bone staple tool wherein the intermediate body and the gripping member are made of a different material.

The bone staple tool wherein the gripping member has a width that is a size of a staple head.

The bone staple tool wherein the channel is off put from a slit center.

The bone staple tool wherein the main body, the intermediate body, and the gripping member are made of one material.

In accordance with an aspect of the invention a bone staple tool comprises a main body having a main body distal end; an intermediate body having an intermediate body proximal end and an intermediate body distal end at least partially disposed within the main body at the intermediate body proximal end, the intermediate body extending from the main body having a slit extending for example from the main body distal end to the intermediate body distal end; and a gripping member that extends from the intermediate body distal end having a first member and a second member defining a channel therebetween; wherein the gripping member is configured to receive a staple no further than a staple bridge top portion.

In accordance with an aspect of the invention a bone staple tool comprises a main body having a main body distal end; an intermediate body having an intermediate body proximal end and an intermediate body distal end at least partially disposed within the main body at the intermediate body proximal end, the intermediate body having a first intermediate body member and a second intermediate body member defining a slit extending from the main body distal end to the intermediate body distal end; and a gripping member that extends from the intermediate body distal end having a first member and a second member extending from the first and second intermediate body members defining a channel therebetween.

In accordance with an aspect of the invention a bone staple tool comprises a main body having a main body distal end; an intermediate body extending from the main body having a slit; and a gripping member that extends from the intermediate body having a first member and a second member defining a channel therebetween, the gripping member being arranged so as to receive and maintain a part of a staple comprising at least two legs and a bridge connecting said at least two legs, the bone staple tool being configured to release the staple by toppling the bone staple tool with a non-zero angle with respect to the staple.

For example the bone staple tool is configured to release the staple by toppling the bone staple tool with a non-zero angle with respect to the staple so that the staple inserter axis forms a non-zero angle, for example an angle comprises between 10° and 60°, for example between 20° and 50°, for example 35°, with a plane comprising staple legs axes when the staple legs extend parallel to each other.

For example the first member and the second member of the gripping member have protrusions configured to retain only an upper portion of the staple bridge within the channel.

For example the bone staple tool comprises an impactor arranged at the proximal end of the bone staple tool.

For example the impactor comprises two lateral walls delimiting a groove and arranged so as to receive the staple bridge within the groove.

In accordance with an aspect of the invention a compression staple comprises at least two legs connected by a bridge, the bridge comprising an upper surface, an opposite lower surface from which the at least two legs extend and two lateral walls connecting the upper and lower surfaces, the bridge further comprising at least one rib arranged on an upper portion of the bridge formed by the bridge upper surface and a part of the bridge lateral walls.

For example, the staple bridge comprises two ribs.

For example, each rib extends on opposite sides of the staple bridge.

For example, each rib extends on an upper part of the bridge lateral walls, the upper part of the bridge lateral walls being linked to the bridge upper surface for example.

In accordance with the present invention, the staple, for example the compression staple, includes, for example is made of, a shape memory material, for example a superelastic shape memory material, such as Nitinol Alloy and the bone staple tool, for example a staple inserter adapted for use with the staple. The staple includes a bridge connecting at least two legs, for example only two legs, for example four legs. The staple is deformable between a first state wherein the at least two legs are substantially non-parallel and a second state wherein said at least two legs are substantially parallel. The staple is configured to reach the second state, when a force, for example an external force, is applied to the staple and to return to the first state, for example by itself, when the force, for example the external force is removed from the staple. The staple can engage the staple inserter, so as to be preloaded on the staple inserter and the staple inserter is configured to cooperate with the staple so as to maintain the staple in its second state until the delivery of the staple into bone of a patient.

The staple inserter includes a staple preloading structural feature (gripping member) that sits entirely above the underside (lower surface) of the staple bridge when the staple is loaded in the staple inserter. This structural arrangement allows the staple to be fully inserted into the bone, to achieve flushness with the external surface of the bone or bones into which the staple is being inserted, while also allowing the staple to be preloaded on the inserter.

The staple inserter allows for flush insertion of the staple on the bone or bones and a disassembly direction of the staple inserter and preloaded staple that does not require rotating of the staple inserter or pulling of the staple inserter to the side of the staple.

The staple inserter clamps the staple so that the staple inserter gripping member is positioned entirely above the lower surface of the staple bridge.

Furthermore, a method of implanting a staple, which uses the staple and staple inserter, can be performed by applying insertion pressure to advance the staple into the bone or bones, releasing the staple from the staple inserter by disengaging the staple gripping member, and retracting the staple inserter to leave the staple implanted in the bones or bones. Advantageously, the structural arrangement of the staple inserter enables the applying and retracting steps to comprise physical movements that occur within the same plane. Indeed, these steps can occur along the same axis, which requires much less space in the surgical field and enables the implantation of staples in areas that might not otherwise readily receive such implantations.

### DRAWINGS

FIG. 1 is a view of a staple preloaded in a second state on a bone staple tool according to a first embodiment.

FIG. 2 is a side view of the staple preloaded in the second state on the bone staple tool according to the first embodiment.

FIG. 3 is a separate view of the staple preloaded in the second state and of the bone staple tool according to the first embodiment.

FIG. 4 is a perspective view of the bone staple tool according to a second embodiment.

FIG. 5 is a section view illustrating the staple preloaded on the bone staple tool according to a third embodiment.

FIG. 6 is a section view illustrating the staple preloaded on a bone staple tool according to a fourth embodiment.

FIG. 7 is a side view of a staple preloaded on a bone staple tool according to fifth embodiment.

FIG. 8 is a perspective view of a staple preloaded on a bone staple tool according to the embodiment of FIG. 7.

FIG. 9 is a view of the bone staple tool according to a sixth embodiment with the staple preloaded before implantation in a bone.

FIG. 10 is a view of the bone staple tool according to the sixth embodiment with the staple preloaded in a second state/deployed position.

FIG. 11 is a view of the bone staple tool according to the sixth embodiment with the staple in the first state after the staple tool main body has been unscrewed from the staple tool intermediate body.

FIG. 12 is a view of an implant in a second state.

FIG. 13 is a view of the implant in a first state.

FIG. 14 is a side view of the implant.

FIG. 15 is a perspective view of the implant in the first state.

FIG. 16 is a perspective view of the bone staple tool intermediate body and gripping member.

FIG. 17 is a view of the bone staple tool intermediate body and gripping member.

FIG. 18 is side view of the bone staple tool intermediate body and gripping member.

FIG. 19 is a perspective view of the bone staple tool main body.

FIG. 20 is a section view of the of the bone staple tool main body.

FIG. 21 is a view of bones prepared for surgery.

FIG. 22 is a view of the staple and bone staple tool according to the sixth embodiment before implantation of the staple in the bones.

FIG. 23 is a view of the staple and bone staple tool according to the sixth embodiment after implantation of the staple in the bones.

FIG. 24 is a view of the main body being unscrewed from the intermediate body and gripping member.

FIG. 25 is a view of the bone staple tool according to the sixth embodiment being removed from the staple.

FIG. 26 is a view of the staple implanted in the bones.

FIG. 27 is a view of the staple implanted in the bones.

FIG. 28 is a view of the staple being impacted by means of the bone staple tool according to the sixth embodiment.

### DETAILED DESCRIPTION

The following description is merely exemplary in nature and is not intended to limit the present disclosure, application or uses. It should be understood that throughout the drawings, corresponding reference numerals indicate like or corresponding parts and features. For simplicity, identical elements are identified by identical reference signs in all figures.

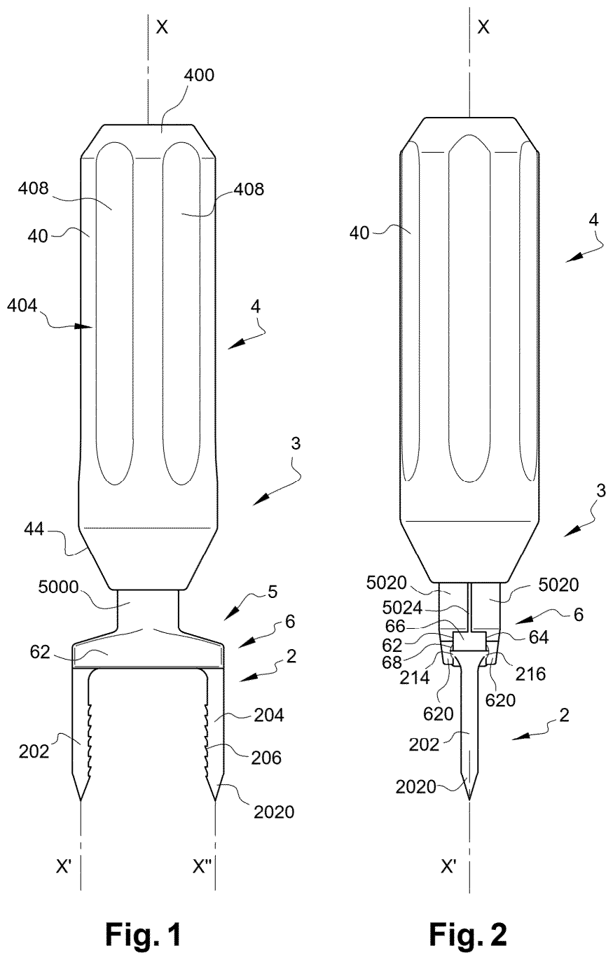

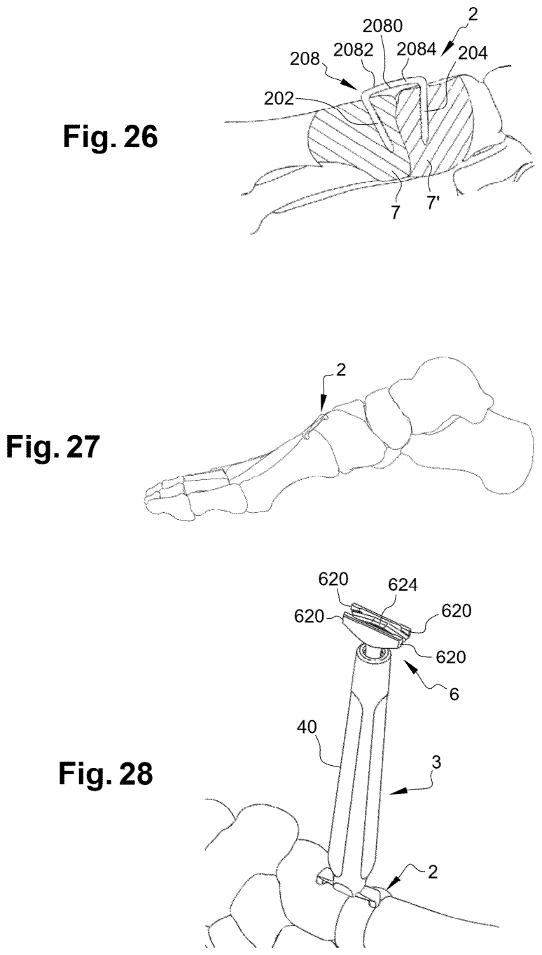

An implant, such as a staple 2, according to the present invention is shown in FIGS. 13 and 15 in a first state (or undeployed state). The staple 2 can include two or more legs, such as the two legs 202, 204 shown in this embodiment. Although two legs 202, 204 are shown in this embodiment, the staple 2 can include any number of legs, for example four legs. The legs 202, 204 are configured to be inserted and maintained into holes 70, 70′ (FIG. 21) drilled in bones 7, 7′ so that the staple 2 is fixed to the bones (FIGS. 26 and 27). To this end, each leg 202, 204, for example each surface of the legs facing each other can include a serration 206 (FIGS. 1, 3, 8, 10, 11, 12, 13), for example including several teeth spaced apart by grooves. Any number of teeth can be on each leg. For example, the serrations 206 can extend over more than half of the surface of each leg 202, 204. Further the length of the legs can be identical or different.

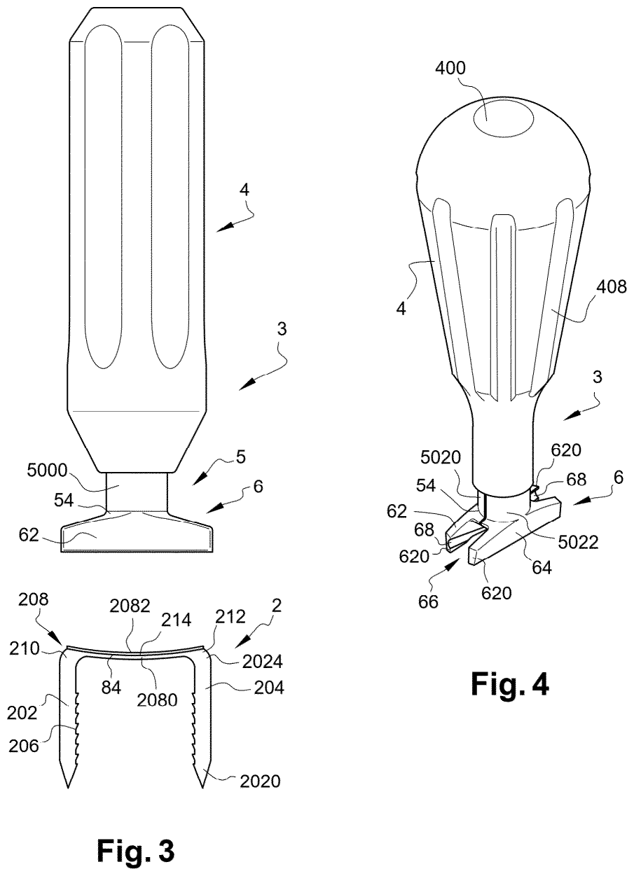

The staple 2 includes a bridge 208 connecting the two or more legs (FIGS. 3 12, 13). In this embodiment wherein the staple includes only two legs, each leg 202, 204 can extend from an end of the bridge 208 so that each leg 202, 204 form with the bridge 208 a corner 210, 212.

When the staples includes four legs, the staple can be arranged so that two of the four legs extend respectively from an end of the bridge 208 to form corners with the bridge 208.

The corners 210, 212, for example each of the two corners 210, 212 of the bridge 208 can be of any radius of curvature suitable for the staple to be inserted in the bone of the patient. Each leg 202, 204 can include a first end 2020 opposite to a second end 2024 attached to the bridge 208. Each of the legs first end 2020 can be tapered for insertion into bone.

The thickness “B” of each leg can be inferior to the thickness “A” of the bridge (FIG. 14). The thickness corresponds for example to the direction that is substantially orthogonal to the main axis of the bridge 208 and to the main axes of leg 202 and/or leg 204, for example of the legs. The thickness corresponds for example to the direction connecting the two lateral opposite walls 2084, 2086 as defined hereafter, for example substantially perpendicular to both such lateral opposite walls 2084, 2086.

The staple bridge 208 can include a lower surface 2080, intended for example to face the bone when the staple is anchored to the bone (FIG. 26), an opposite upper surface 2082 and two lateral opposite walls 2084, 2086 arranged between, for example connecting the lower surface 2080 and the upper surface 2082 (FIG. 14).

The staple bridge 208 can include at least one rib, for example two ribs 214, 216, arranged on an upper or top portion of the staple bridge 208 (FIGS. 2, 3, 5, 6, 7, 14, 15). The staple bridge upper portion can includes the staple bridge upper surface 2082 and an upper portion of the staple bridge lateral walls 2084, 2086. Each rib 214, 216 can extend on a side of the staple bridge upper portion, for example on an opposite side of the staple bridge upper portion. For example, each rib 214, 216 can extend on upper parts 218, 220 (FIG. 14) of the staple bridge lateral wall 2084, 2086. The upper part of the lateral wall 2084, 2086 can be linked to the upper surface 2082 of the staple bridge 208.

As illustrated in FIG. 14, each side of the staple bridge 208 can comprise from the lower surface 2080 to the upper surface 2082 of the staple bridge 208, a lower part 218′, 220′ of the lateral walls 2084, 2086 and then a rib 214, 216 arranged for example on an upper part 218, 220 of the lateral wall 2084, 2086. The rib 214, 216 is protruding from the respective lateral wall, for example compared to the respective lower part 218′, 220′ of the respective lateral walls 2084, 2086.

The ribs 214, 216 can share a complementary form with the part of the bone staple tool 3 intended to retain the staple as this will be described later in the specification.

The bridge 208 and the legs 202, 204 of the staple 2 can be made in one single piece.

The staple 2 can include, for example can consist of, for example can be made of, an elastic material, for example a shape memory material, for example a shape memory material comprising superelastic properties, for example Nitinol alloy. For example, the staple can be made of Nitinol alloy.

In the staple first state (or undeployed state) shown in FIGS. 13 and 15, the staple legs 202, 204 extend non-parallel to each other. This state corresponds to the natural shape of the staple 2, for example a rest state, for example an unconstrained state.

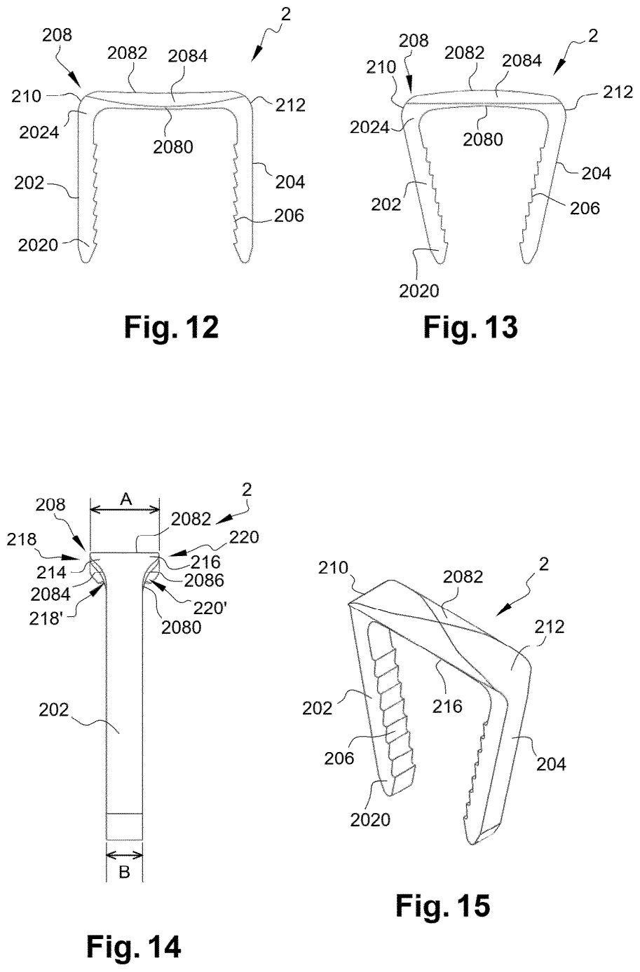

However, for the staple 2 to be implanted in a bone, the staple legs 202, 204 have to extend parallel to each other. To this end, a constraint, for example a mechanical constraint can be applied to the staple 2 so that the staple legs 202, 204 move away from each other, toward a position wherein the legs 202, 204 extend parallel to each other. The staple 2 can be configured to be deformed from the first state (or undeployed state), wherein the legs extend non-parallel to each other, to a second state (or deployed state), wherein the legs extend parallel to each other. The deformation can occur when a force, for example an external force, is applied to the staple 2. Any suitable tool known from the art, such as a retractor (not represented) for example, can be used to drive the staple legs parallel to each other. In this second state or deployed state where the legs extend parallel to each other, the bridge 208 can be deformed to a concave shape to accommodate the positions of the legs (FIGS. 3 and 12), the concavity being for instance located above the upper surface according to the direction defined from the lower surface to the upper surface.

FIGS. 1, 3, 12 and 14 show the staple 2 in the second state wherein the legs 202, 204 extend parallel to each other. In this state, the staple 2 can be implanted into holes 70, 70′ drilled in bones 7, 7′. Once the staple 2 has been deformed to the second state, for example by means of the retractor, the staple 2 can be preloaded on the bone staple tool 3 in order to be implanted in the bone. The bone staple tool 3 can be configured to maintain the staple 2 in the second state, when the staple is preloaded on the bone staple tool 3, for example by applying a force on the staple. The staple 2 is configured to remain in the second state until the external force, for example the force generated by the bone staple tool 3, is removed from the staple 2.

The staple 2 can be further configured to return to its first state, for example by itself, for example when the external force, for example the bone staple tool 3 force, ceases to be applied to the staple. Due to the superelastic properties of the staple shape memory material, the staple legs 202, 204 can return to their natural shape and converge toward each other so that the staple 2 returns to its first state. In the first state (or undeployed state) the bridge 208 can retain a convex shape, as opposed to the concave shape of the second state, the convexity facing for example substantially up, to accommodate the positions of the legs 202, 204 (FIG. 13).

The bone staple tool or staple inserter 3 is now described. The staple inserter 3 is configured to cooperate with the staple 2 so as to maintain the staple in its second state until the delivery of the staple into bone. The staple 2 can be preloaded in the staple inserter 3 after the staple has been deformed toward the second state, for example by means of the retractor. The staple preloaded on the staple inserter in its second state can be packaged prior to be used by a surgeon.

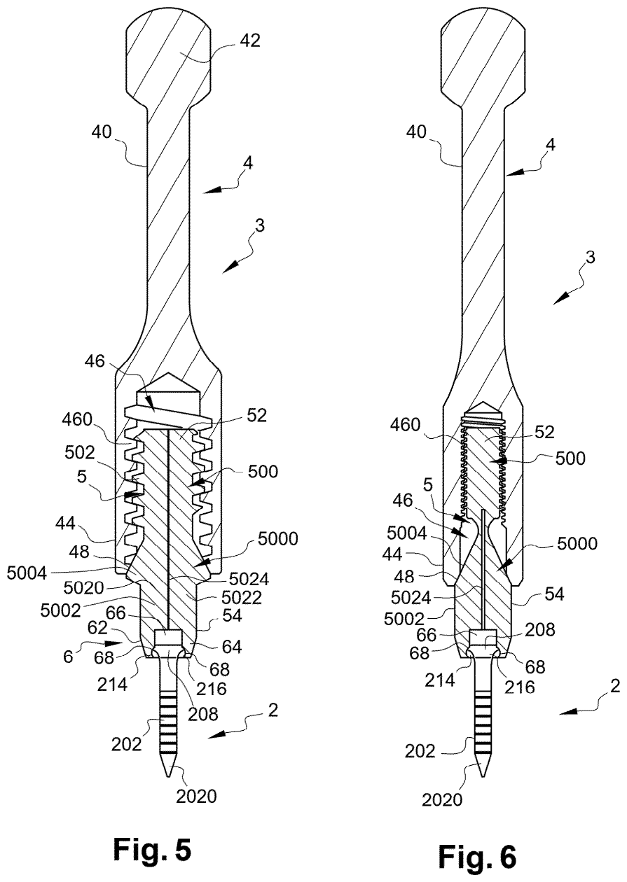

The staple inserter 3 can include a main body 4 having a main body proximal end 42 and a main body distal end 44. The staple inserter 3 can include an intermediate body 5 having an intermediate body proximal end 52 and an intermediate body distal end 54 (FIGS. 5, 6). The intermediate body 5 can be, at least partially, disposed within the main body 4 at the intermediate body proximal side (FIGS. 5, 6). The intermediate body 5 can have a first intermediate body member 5020 and a second intermediate body member 5022 defining a slit 5024 (FIGS. 4, 5, 6). The staple inserter 3 can include a gripping member 6 extending from the intermediate body distal end 54. The gripping member 6 can include a first member 62 and a second member 64 extending respectively from the first and second intermediate body members 5020, 5022 and defining a channel 66 therebetween (FIGS. 2, 4, 5, 6, 16, 18).

The intermediate body 5 and the gripping member 6 can be made in one single piece. For example, the intermediate body 5 and the gripping member 6 can be made of metal.

The intermediate body 5 is now described (FIGS. 5, 6, 16, 17). The intermediate body 5 can include a first part 500 and a second part 5000 which can be made in one single piece. The intermediate body first part 500 can include the intermediate body proximal end 52 and can have a thread 502 so that the intermediate body 5 can be fastened to the main body 4, for example screwed within the main body (FIGS. 5, 6, 16, 17, 18). The intermediate body first part 500 can be cylindrical and is intended to be disposed within the main body. When the intermediate body 5 and the main body 4 are screwed together, the main body can surround the intermediate body first part 500 (FIGS. 5, 6).

The second part 5000 of the intermediate body 5 can extend from the intermediate body first part 500. The intermediate body second part 5000 can include the intermediate body distal end 54 and can comprise a portion, for example a first portion, which is cylindrical 5002. The intermediate body second part 5000 is intended to be disposed at least partially outside of the main body 4. The intermediate body second part 5000 can be slotted, that is to say that the intermediate body second part 5000 can comprise the slit 5024. As previously described, the intermediate body second part 5000 can include a first intermediate body member 5020 and a second intermediate body member 5022 defining the slit 5024 (FIGS. 5, 6, 16, 17, 18). The slit 5024 can extend from the intermediate body proximal end 52 to the intermediate body distal end 54 (FIG. 5), or, for example, from the intermediate body first part 500 to the intermediate body distal end 54 (FIGS. 6, 16, 17, 18).

The intermediate body slit 5024 is configured to allow the first and second intermediate body members 5020, 5022 to be movable relative to each other, for example to converge toward each other, for example with a spring effect, for example when a force, for example an external force is applied to the intermediate body second part. The intermediate body slit 5024 allows the first and second intermediate body members 5020, 5022 to move toward each other, for example when the external force is applied to the intermediate body second part.

This external force can be generated by the main body 4 for example. To this end the intermediate body second part 5000 can include a portion, for example a second portion 5004, which can be conical and arranged to enable the main body 4 to come into contact with the intermediate body second part 5000 so as to apply a force on the intermediate body second part 5000 (FIG. 10).

The main body 4 can be configured to apply a force on the intermediate body 5, for example when the main body 4 comes into contact with the intermediate body conical portion 5004, for example when the main body 4 and the intermediate body 5 are fastened together, so as to apply a force on the intermediate body second part 5000 and to move the first and second intermediate body members 5020, 5022 toward each other (FIGS. 5, 6, 10 16, 17, 18) as it will be described in the subsequent paragraphs.

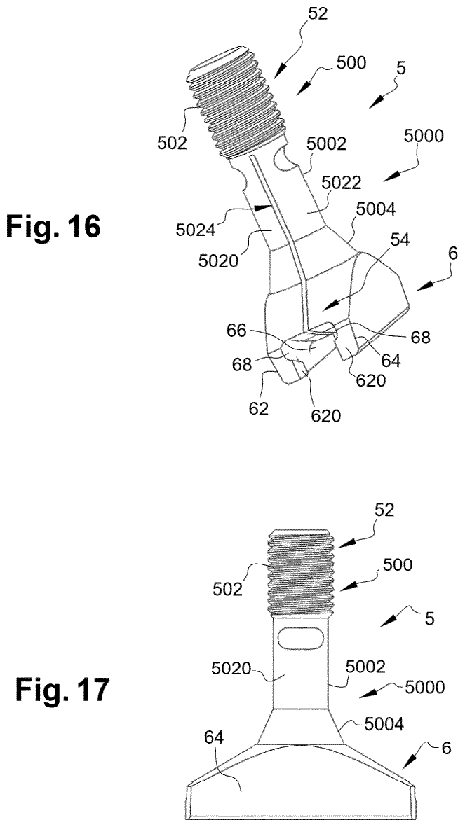

The first and second intermediate body members 5020, 5022 can be further configured to return to their natural shape, for example by themselves, for example by moving away from each other, for example when the external force is removed from the intermediate body (FIG. 16).

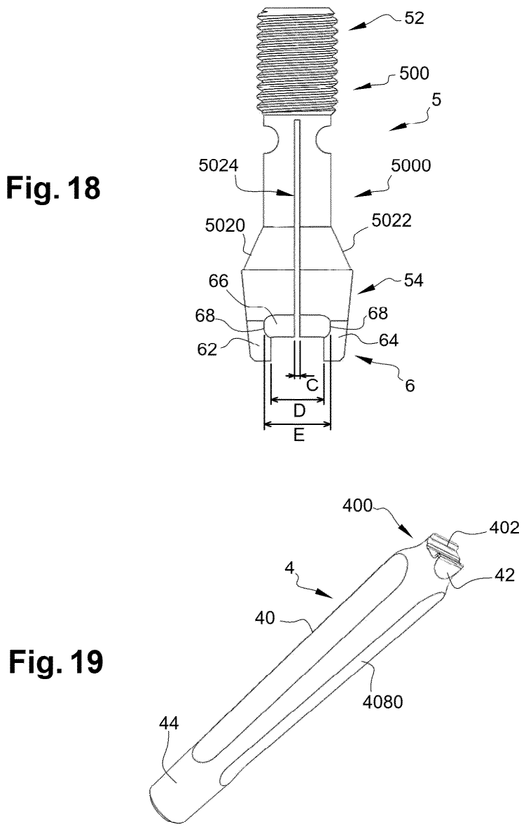

When the first and second intermediate body members 5020, 5022 are in their natural shapes, for example when no external force is applied on the first and second intermediate body members, the width “C” of the slit 5024 can be of 0.4 mm (FIG. 18).

The width “C” corresponds for example to the direction that is substantially orthogonal to the plane defined by the slit 5024. The width “C” corresponds for example to the direction orthogonal to the main axes of the first and second intermediate body members. The width corresponds for example to the direction connecting the first and second intermediate body members, for example the direction substantially perpendicular to the facing surfaces of both such first and second intermediate body members.

The gripping member 6 of the staple inserter 3 is now described.

The gripping member first and second members 60, 62 extend respectively from the intermediate body first and second intermediate body members 5020, 5022 (FIGS. 2, 4, 5, 6, 16). As illustrated in FIGS. 2, 5, 6, 16 and 18, the intermediate body slit 5024 can open on the channel 66 formed between the gripping member first and second members 62, 64. The width “D” of the channel can be superior to the width “C” of the slit (FIG. 18).

The width “D” is for example parallel to the direction defined by width “C”. The width “D” corresponds for example to the direction that is substantially orthogonal to the plane defined by the slit 5024. The width “D” corresponds for example to the direction orthogonal to the main axes of the gripping member first and second members 60, 62. The width “D” corresponds for example to the direction connecting the gripping member first and second members 60, 62, for example the direction substantially perpendicular to the facing surfaces of the protrusions as defined hereafter of both such gripping member first and second members 60, 62. The width “D” corresponds for example to the same direction as the thickness of the staple when the staple 2 is preloaded on the staple inserter 3.

The gripping member 6 can form a staple interface, such as a jaw, configured to hold the staple 2, in its second state until the delivery of the staple into bone of a patient (FIGS. 1, 10). The gripping member 6 can be configured to be in direct contact with the staple 2.

The gripping member 6 is arranged so that the gripping member first and second members 62, 64 can hold and come into contact with the upper portion of the staple bridge 208 only (FIGS. 2, 5, 6).

The gripping member first and second members 62, 64 can each present a groove 68, arranged respectively on the surfaces of first and second members facing each other (FIGS. 2, 4, 5, 6, 16, 18). Each of the grooves 68 can open on the channel 66 formed between the gripping member first and second members 62, 64. The width “E” between the surfaces of the first and second members where the grooves are arranged is superior to the width “D” of the channel 66 formed between the first and second members, the width “D” of the channel 66 formed between the first and second members being superior to the width of the slit 5024 (FIG. 18).

The width “E” is for example parallel to the direction defined by width “E”. The width “E” corresponds for example to the direction that is substantially orthogonal to the plane defined by the slit 5024. The width “E” corresponds for example to the direction orthogonal to the main axes of the gripping member first and second members 60, 62. The width “E” corresponds for example to the direction connecting the gripping member first and second members 60, 62, for example the direction substantially perpendicular to the facing surfaces of the grooves of both such gripping member first and second members 60, 62. The width “E” corresponds for example to the same direction as the thickness of the staple when the staple 2 is preloaded on the staple inserter 3.

Each groove 68 can form a housing configured to receive and hold a rib of the staple bridge. The grooves 68 share a complementary shape with the ribs 214, 216 of the staple bridge 208 (FIGS. 2, 5, 6).

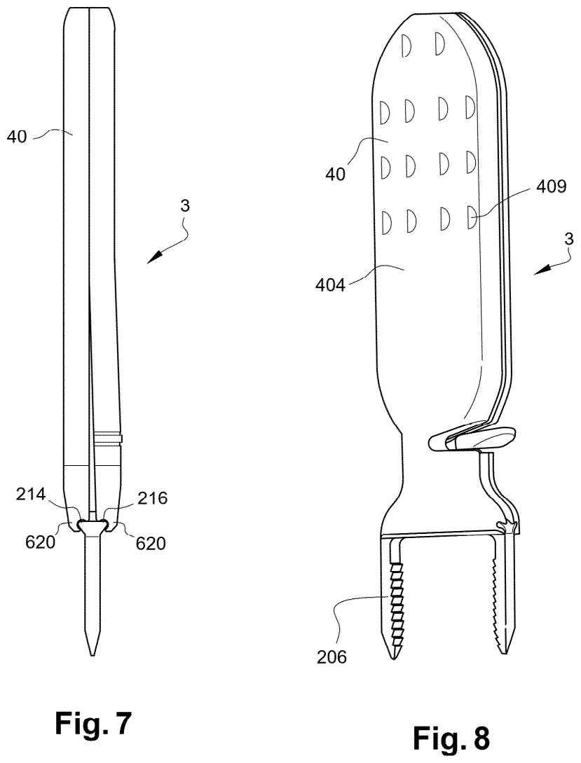

Each surface of the first and second members 62, 64 facing each other can include two protrusions 620 (FIGS. 4, 7, 28). Each protrusion can form a shoulder arranged to engage the underside of the staple bridge ribs 214, 216 and to hold the staple in the deployed state. In the example, the gripping member comprises four protrusions 620 (FIGS. 7 and 28). Said protrusions can delimit at least a part of the gripping member first and second members 62, 64 grooves 68.

The gripping member 6 first and second member 62, 64 can each further include an inner surface arranged to be in contact with the staple bridge upper surface 2082, for example a central portion of the bridge upper surface 2082. Said surfaces can each include a projecting part 624 (FIG. 28), arranged for example in the middle of each of said surface and engaging the staple bridge upper surface 2082 so as to maintain the bridge concave to accommodate the positions of the legs.

The main body 4 is now described. The main body 4 can include a handle 40 (or grip) configured to be graspable by a user's hand such as a surgeon's hand. The handle 40 can be of any suitable shape such as an elongated shape (first, fifth and sixth staple inserter embodiments (FIGS. 1, 7 and 9)), an elongated shape ended with a rounded body (third and fourth staple inserter embodiments (FIGS. 5, 6)), or a pear shape (second staple inserter embodiment (FIG. 4)).

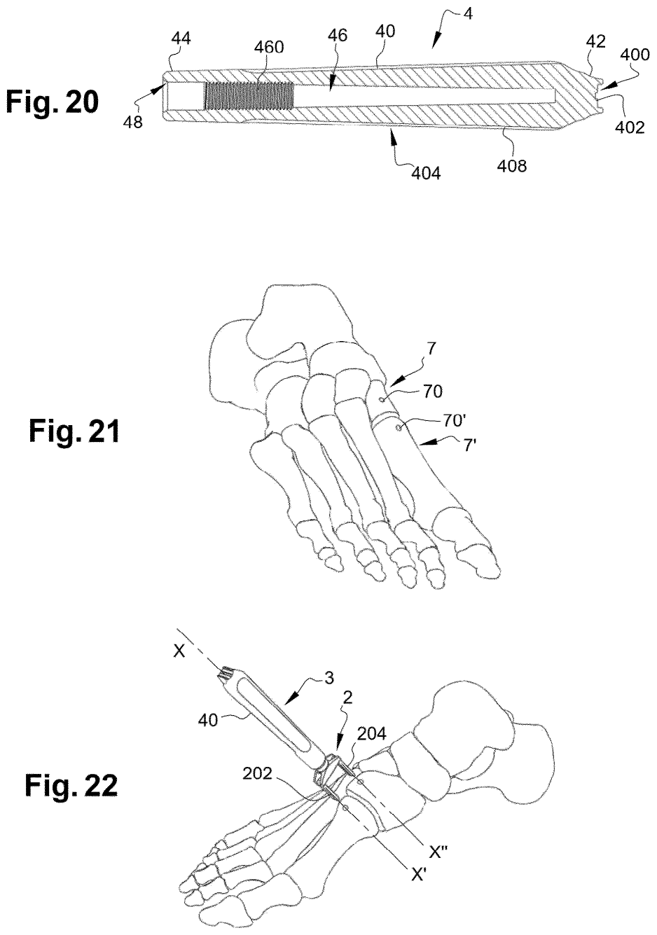

The handle 4 can have a bore 46 (FIGS. 5, 6, 20), part of which is threaded 460 so as to cooperate with the intermediate body threaded 502 first part 500. The intermediate body 5 can thus be fastened, for example screwed, within the handle 40 of the main body. The distal end 44 of the handle 40, for example the surface of the handle defining the bore 46 at the distal end 44 of the handle, has a conical portion 48, defining an opening which compelets the intermediate body second part conical portion 5004, configured to come into contact with the intermediate body second part conical portion 5004, for example when the intermediate body is further screwed to the handle.

The conical portion 48 of the handle 40 is configured to come into contact with the conical portion 5004 of the intermediate body second part 5000 (FIGS. 5, 6), when the intermediate body is further screwed with the main body handle. This configuration allows the handle to apply a force on the intermediate body (by contact between the conical portions) so as to move the first and second intermediate body members toward each other and thus the gripping member first and second members toward each other. In this configuration, the staple is gripped between the gripping member first and second members.

According to another embodiment illustrated in FIGS. 7 and 8, the bone staple tool 3 can comprise the main body, the intermediate body and the gripping member which can be made in one single piece. The bone staple tool can further comprise an actuation means, for example a wedge configured, when it is actuated and mounted on the bone staple tool, to maintain the gripping member first and second members of the gripping member closer together. The gripping member first and second members of the gripping member can further move away from each other when the actuation means is removed from the staple tool.

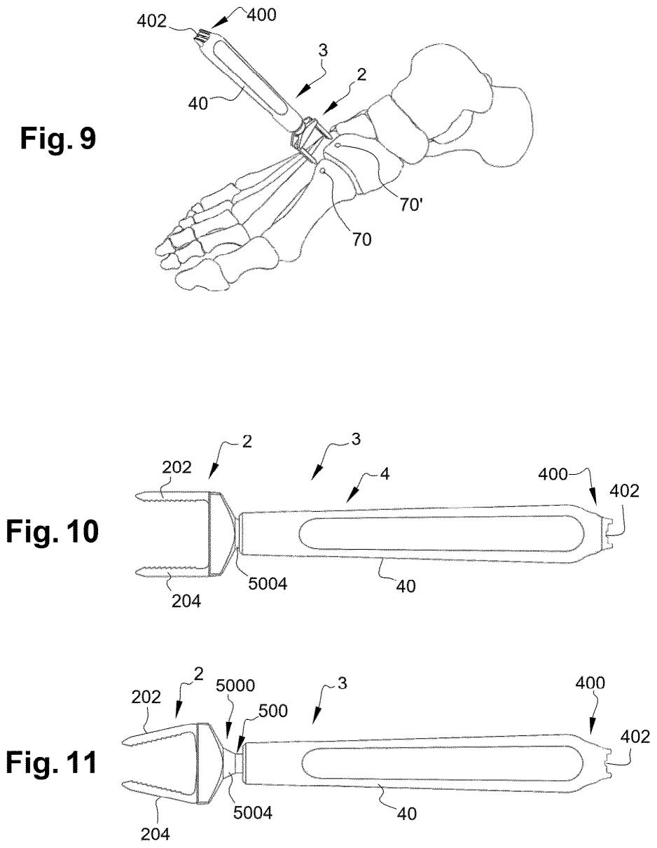

The handle opposite end 42, for example the handle proximal end, can include an impactor 400 (FIGS. 9, 10 19, 28), designed to be complementary with the staple bridge 208 shape and dimension. The impactor can comprise two lateral walls delimiting a groove 402. The lateral walls delimiting are arranged so as to receive the staple bridge 208 within the groove. The handle impactor allows for a final staple impaction with the handle by the surgeon if needed (FIG. 28). For example, the handle impactor is arranged so as to come into contact with the upper surface of the staple bridge in order to allow final staple impaction with the handle by the surgeon if needed.

Gripping part can be arranged on the handle, for example on the lateral parts 404 of the handle so as to improve the handle ergonomics

As illustrated in the first (FIG. 1 to 3) and second (FIG. 4) embodiments of the staple inserter, the handle 40, for example the lateral parts 404 of the handle can have a plurality of slots 408, extending for example along a longitudinal axis “X” of the handle 40.

As illustrated in the fifth embodiment of the staple inserter (FIG. 8), the handle 40, for example the lateral parts 404 of the handle can include a plurality of 409 excrescences for example circular.

As illustrated in the sixth embodiment of the staple inserter (FIG. 19), the handle 40, for example the lateral parts 404 of the handle can have flat portions or flat spots 4080.

The preloading of the staple on the staple inserter can be performed before packaging and requires the following steps.

First, the staple 2 in the first state (or undeployed position) (FIG. 13) is moved to the second state (or deployed position) (FIG. 12) by means of a specific and dedicated tool, for example a retractor (not represented).

The intermediate body slit 5024 of the staple inserter 3 allows the first and second intermediate body members 5020, 5022, and thus the gripping member first and second members 62, 64, which extend thereof, to be arranged so that the staple can slide onto and off the gripping member, when no external force is applied to the intermediate body (FIG. 18).

At this stage, the intermediate body first part can be, at least partially, already arranged within the handle and screwed within the handle.

The staple bridge 208 is then inserted in the gripping member 6 (FIGS. 2, 5, 6, 7). Both the staple bridge 208 shape (for example the staple bridge ribs shape 214, 216) and the gripping member 6 shape (for example the gripping member grooves 68 and protrusions 620) complement each other and are unique for each staple size. All dimensions (width of the slit 5024, the channel 66, the grooves 68) are configured together so as to create complementarity between the staple 2 and the gripping member 6 when staple, in second state is assembled with the staple inserter 3.

The intermediate body 5 can be further screwed in the handle threaded bore 46 so that the handle 40 moves until the handle conical portion 48 comes in contact with the intermediate body second part conical portion 5004 (FIGS. 5, 6, 10).

The contact between the conical portions 48, 5004, of the handle and the intermediate body allows the handle 40 to apply a force on the intermediate body 5 so that the member first and second members 62, 64 converge toward each other and hold the staple 2 therebetween, in the deployed state.

Thanks to the slit 5024 of the intermediate body 5, the pressure exerted over the intermediate body (by the contact between the conical portion 48 of the handle 40 and the conical portion 5024 of the intermediate body second part 5000) causes a spring effect so as to bring the first and second intermediate body members 5020, 5022 closer to each other and thus bring the gripping member first and second members 62, 64 closer to each other. Further screwing thus permits to block the staple between the gripping member first and second members 62, 64.

The spring effect is given by the width of the slit, of for example comprised between 0.2 and 0.6 mm, for example 0.4 mm, and the contact between both conical portion 48 of the handle, such conical portion 48 having for example a minimum diameter for example comprised between 4 and 8 mm, for example 6.1 mm, a cone angle of the conical part 48 being for example comprised between 15 and 45°, for example between 20 and 30°, for example 25°, and the conical portion 5004 of the intermediate body second part, such conical portion 5004 having for example a minimum diameter for example comprised between 3 and 7 mm, for example 5 mm, a cone angle of the conical part 5004 being for example comprised between 15 and 45°, for example between 20 and 30°, for example 25°.

This spring effect as well as the threaded portion of the intermediate body first part and the handle allow to grip the staple, maintain and secure it in the deployed state.

After the preloading of the staple 2 on the staple inserter 3, the under surface of the gripping part 6 is always above the lower surface 2080 of the staple bridge 208 to allow the insertion of the staple directly flush with the bone as the gripping part does not interfere during staple insertion. There is no clearance between bones and staple.

The staple bridge ribs 214, 216 can extend in the gripping member grooves 68 and the gripping member protrusions 620 can extend on the underside of the staple bridge ribs 214, 216 so as to maintain the staple 2 in its deployed position.

This configuration, facilitates the release of the staple form the staple inserter once the staple has been implanted in the bone. The staple inserter does not need to be separated from the staple by a twist or pushed to the side of the staple because the gripping member does not extend on the lower surface of the staple bridge. The staple inserter can be released from the staple by toppling the staple inserter with respect to the staple so that the staple inserter axis form a non-zero angle with a plane comprising the staple legs axes when the staple legs extend parallel to each other and by pulling the staple inserter from the staple, for example by rotating the staple inserter with respect to the main axis of the bridge.

FIGS. 1 and 2 show the staple 2 engaged or preloaded on the staple inserter. In this position, the main body extend along a main body axis “X”, for example longitudinal axis, which is parallel to the staple legs axes “X′”, “X′”, for example the staple legs longitudinal axis, when the staple legs extend parallel to each other.

FIG. 25 show the staple inserter 3 released from the staple. According to the configuration of the invention, the staple inserter 3 is configured to be disengaged from the staple by toppling the staple inserter 3 with respect to the staple 2 so that the staple inserter axis “X” forms a non-zero angle, for example an angle comprises between 20° and 60°, for example 45°, with a plane comprising the staple legs axes “X′”, “X″” when the staple legs 202, 204 extend parallel to each other. When toppling the staple inserter with respect to the staple, the protrusions 620 of the gripping member first and second members 62, 64 disengage the ribs 214, 216 of the staple bridge 208. The staple inserter can then be pulled from the staple.

The advantages of the disclosure are to allow direct insertion of the staple flush to bones thanks to the design shape of the gripping part. The staple inserter, preloaded with the staple can be packaged prior to be used by the surgeon to implant the staple in bone.

Another advantage is to reduce skin incision as there is no need to twist or extend on the side of the staple inserter to remove the staple inserter after staple release.

Furthermore, a method of implanting a staple, which uses the staple and staple inserter, can be performed by applying insertion pressure to advance the staple into the bone or bones, releasing the staple from the staple inserter by disengaging the staple preloading structural feature (gripping member), and retracting the staple inserter to leave the staple implanted in the bones or bones. Advantageously, the structural arrangement of the staple inserter enables the applying and retracting steps to comprise physical movements that occur within the same plane. Indeed, these steps can occur along the same axis, which requires much less space in the surgical field and enables the implantation of staples in areas that might not otherwise readily receive such implantations.

FIGS. 21 to 28 illustrate a method for implanting a staple according to the invention.

FIG. 21 illustrates a first step of the method consisting in bone preparation. An incision of a few millimeters to a few centimeters corresponding to the staple size chosen for implantation is created, for example using a scalpel. Then, the osteotomy is created and/or the fusion site needed to implant the staple is prepared. Finally, bone preparation is completed by drilling holes 70, 70′ into bones 7, 7′ corresponding to the distance between staple legs of the staple chosen for implantation.

FIG. 22. illustrates the next step consisting of taking out the staple inserter 3, including the staple 2 preloaded in its second state (deployed state), from its packaging and presenting it in front of prepared holes 70, 70′ ready to receive staple legs 202, 204. The main body axis “X” extend parallel to the staple legs axes “X′”,“X″”.

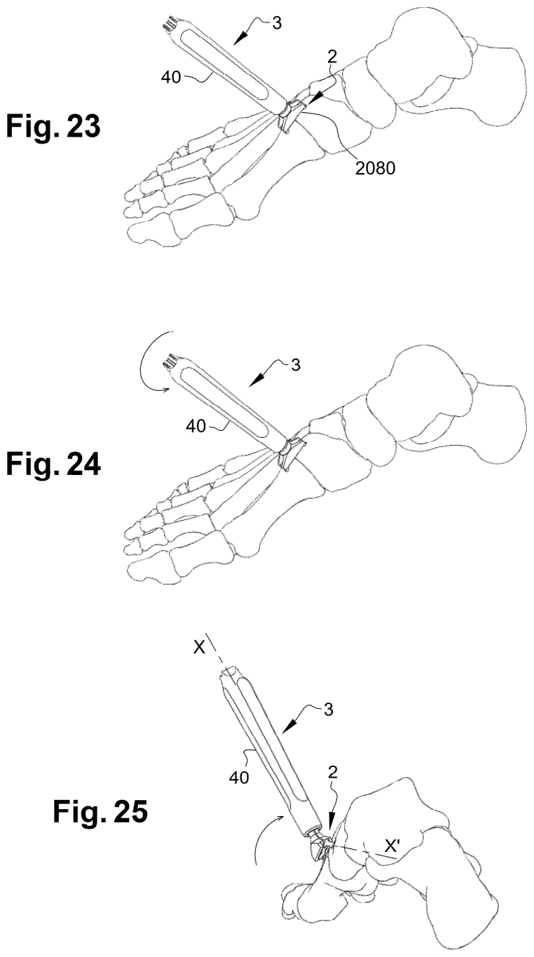

FIG. 23 illustrates the next step wherein the staple 2 is inserted into the bones until staple bridge lower surface comes in contact with the bone.

FIG. 24 illustrates the next step. At this stage the staple can be released from the staple inserter by turning the handle 40 from 1 to 10 turns in anti-clockwise direction. By unscrewing, the handle moves up along the intermediate body threaded first part (FIG. 11).

The unscrewing action allows to disengage the conical portion 48 of the handle, from the conical portion 5004 of the intermediate body second part which release the spring effect of the slit 2024 (FIG. 11).

FIG. 25 illustrates the next step where the unscrewing action allows to remove the staple 2 from the staple inserter 3 by performing a toppling action of approximately 45° from the axis of the staple legs and simultaneously pulling over the handle in the direction of its own axis.

FIGS. 11, 26 and 27 illustrate the staple after the staple inserter has been released from the staple. The staple is flush with the bone. As there is no external force, for example mechanical maintain of the staple in second state anymore (deployed state), the staple returns to the first state, for example by itself. The staple naturally starts its compression action by coming back in the first state (undeployed state). Due to the superelastic properties of the shape memory material of the staple, the legs can converge toward each other for example when the force is removed from the staple.

If the surgeon wants to perform a final impaction at some point, the staple inserter impactor (groove arranged at the main body or handle proximal end) can be used (FIG. 28).

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular examples disclosed herein have been selected by the inventors simply to describe and illustrate examples of the invention and are not intended to limit the scope of the invention or its protection, which is to be given the full breadth of the appended claims and any and all equivalents thereof, including all claims made in all procedurally related applications.

What is claimed is:

1. A bone staple tool (3) comprising:
a main body (4) having a main body distal end
an intermediate body (5) having an intermediate body proximal end (52) and an intermediate body distal end (54) at least partially disposed within the main body (4) at the intermediate body proximal end, the intermediate body (5) extending from the main body having a slit (5024); and
a gripping member (6) that extends from the intermediate body distal end (54) having a first member (62) and a second member (64) defining a channel (66) therebetween.

2. The bone staple tool (3) of claim 1, wherein a width of the main body (4) is larger than a width of the intermediate body (5).

3. The bone staple tool (3) of claim 1, wherein the channel (66) is substantially centered at the slit (5024).

4. The bone staple tool (3) of claim 1, wherein the first member (62) and the second member (64) of the gripping member (6) have protrusions (620) configured to retain a staple within the channel (66).

5. The bone staple tool (3) of claim 1, wherein the gripping member (6) is configured to receive a staple (2) no further than a staple bridge (208) top portion.

6. The bone staple tool (3) of claim 4, further comprising a mechanism (48) for retracting the protrusions (620).

7. The bone staple tool (3) of claim 1, wherein the main body (4) has a grip (40).

8. The bone staple tool (3) of claim 1, wherein a width of the gripping member (6) is larger than a width of the intermediate body (5) and the main body (4).

9. The bone staple tool (3) of claim 1, wherein the channel (66) has a channel width that is smaller than a head of a staple.

10. The bone staple tool (3) of claim 1, wherein the intermediate body (5) is a metal.

11. The bone staple tool (3) of claim 1, wherein the main body (4) is a polymer.

12. The bone staple tool (3) of claim 1, wherein the gripping member (6) is a metal.

13. The bone staple tool (3) of claim 1, wherein the gripping member (6) has a gripping member distal end and a gripping member proximal end, and the gripping member proximal end has a width more narrow than the gripping member distal end.

14. The bone staple tool (3) of claim 1, wherein the intermediate body (5) and the gripping member (6) are made of the same material.

15. The bone staple tool (3) of claim 1, wherein the intermediate body (5) and the gripping member (6) are made of a different material.

16. The bone staple tool (3) of claim 1, wherein the gripping member (6) has a width that is a size of a staple head.

17. The bone staple tool (3) of claim 1, wherein the channel is off put from a slit center.

18. The bone staple tool (3) of claim 1, wherein the main body (4), the intermediate body (5), and the gripping member (6) are made of one material.

19. The bone staple tool (3) of claim 1, wherein the slit (5024) extends from the main body distal end to the intermediate body distal end.

20. A bone staple tool (3) comprising:
a main body (4) having a main body distal end (44);
an intermediate body (5) extending from the main body (4) having a slit (5024); and
a gripping member (6) that extends from the intermediate body (5) having a first member (62) and a second member (64) defining a channel (66) therebetween, the gripping member (6) being arranged so as to receive and maintain a part of a staple (2) comprising at least two legs (202, 204) and a bridge (208) connecting said at least two legs (202, 204),
the bone staple tool (3) being configured to release the staple (2) by toppling the bone staple tool (3) with a non-zero angle with respect to the staple (2).

21. The bone staple tool (3) according to claim 20 wherein the bone staple tool (3) is configured to release the staple (2) by toppling the bone staple tool (3) with a non-zero angle with respect to the staple (2) so that the a staple inserter axis forms a non-zero angle, for example an angle comprises between 20° and 60°, for example 45°, with a plane comprising staple legs axes when the staple legs extend parallel to each other.

22. The bone staple tool (3) according to claim 21 wherein the first member (62) and the second member (64) of the gripping member (6) have protrusions (620) configured to retain only an upper portion of the staple bridge (208) within the channel (66).

23. The bone staple tool (3) according to claim 21 wherein the bone staple tool (3) comprises an impactor (400) arranged at the proximal end (42) of the bone staple tool.

24. The bone staple tool (3) according to claim 23 wherein the impactor (400) comprises two lateral walls delimiting a groove (402) and arranged so as to receive the staple bridge (208) within the groove (402).

25. A compression staple (2) comprising at least two legs (202, 204) connected by a bridge (208), the bridge (208) comprising an upper surface (2082), an opposite lower surface (2080) from which the at least two legs (202, 204) extend and two lateral walls (2084, 2086) connecting the upper and lower surfaces, the bridge (208) further comprising at least one rib (214, 216) arranged on an upper portion of the bridge (208), said upper portion being formed by the bridge upper surface and a part of the bridge two lateral walls.

26. The compression staple (2) according to claim 25 wherein the staple bridge (208) comprises two ribs (214, 216).

27. The compression staple (2) according to claim 26 wherein each of the ribs (214, 216) extends on opposite sides of the staple bridge (208).

28. The compression staple (2) according to claim 26 wherein each of the ribs (214, 216) extends respectively on an upper part of a bridge staple lateral wall (2084, 2086).
